(19) United States
(12) Patent Application Publication
(10) Pub. No.: US 2009/0214597 A1
(43) Pub. Date: Aug. 27, 2009

(54) NON-INJECTION IMMUNOTHERAPY

(75) Inventors: Peter Moldt, Rungsted Kyst (DK); Ove Pedersen, Ringsted (DK); Klaus Theobald, Paoli, PA (US); Michael Flanagan, Kalamazoo, MI (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Curalogic A/S, Kobenhavn K (DK)

(21) Appl. No.: 11/915,564

(22) PCT Filed: May 19, 2006
(86) PCT No.: PCT/US06/19374

§ 371 Date: Jul. 15, 2008

(30) Foreign Application Priority Data
May 27, 2005 (US) 11140457

(51) Int. Cl. A61K 39/36; A61P 37/08

(52) U.S. Cl. 424/275.1

(57) ABSTRACT

Methods and compositions are disclosed for the reduction of immune responses to an exogenous allergen. The reduction is obtained by pre-treatment or pre-conditioning, without injection, of a subject with the allergen so as to reduce immune responses in the subject upon additional exposure to the allergen. The invention is advantageously used in a variety of contexts, including seasonal allergies wherein pre-treatment or pre-conditioning is performed before the onset of the seasonal allergy.

### RELATED APPLICATIONS

This application claims benefit of priority to U.S. patent application Ser. No. 11/140,457, filed May 27, 2005, which is hereby incorporated by reference as if fully set forth.

### FIELD OF THE INVENTION

This invention relates to the reduction of immune responses to an exogenous allergen. The reduction is obtained by pre-treatment or conditioning, without injection, of a subject with the allergen so as to reduce immune responses in the subject upon environmental exposure to the allergen. The invention may be advantageously used in a variety of contexts, including seasonal allergies wherein pre-treatment or conditioning is performed before the onset of the seasonal allergy.

### BACKGROUND OF THE INVENTION

Immunotherapy has the goal of modifying allergic immune responses. Various routes of administering allergens have been utilized in immunotherapy efforts, resulting in a selection for high dose, subcutaneous immunotherapy (SCIT) as a standard treatment to treat allergies (see ref. 3 herein). Administration by injection delivers an intact agent into the body where needed, without the possibility of degradation during passage through the digestive system.

This is in contrast to non-injection routes, which include oral immunotherapy (OIT), sublingual immunotherapy (SLIT), sublingual spit immunotherapy (SLIT-spit), and sublingual swallow immunotherapy (SLIT-swallow). Oral administration must be conducted with awareness of the need for an agent to pass through the digestive tract and be absorbed into the body without destruction.

Experience with administration of immunotherapy has included a buildup (pre-treatment) phase and a maintenance phase with allergens. In cases of treatment of seasonal allergies the buildup (pre-treatment) phase has been conducted prior to the start of the season for the allergen in question. Pre-treatment with allergens has been performed with a focus on safety considerations in order to minimize the risk of systemic reactions. When an allergen is used to treat an allergy, a pretreatment period with increasing dosages is used to reduce the likelihood of undesirable and potential lethal side effects seen in cases of starting with a maintenance dose (ref 40 herein).

Citation of documents herein is not intended as an admission that any is pertinent prior art. All statements as to the date or representation as to the contents of documents is based on the information available to the applicant and does not constitute any admission as to the correctness of the dates or contents of the documents.

### BRIEF SUMMARY OF THE INVENTION

This invention provides for the reduction of immune responses to an exogenous allergen and is based in part on the discovery that pre-treatment, such as treatment before the period of a seasonal allergy, of at least 8 weeks is essential to obtain efficacy. The reduction is mediated by administration of the allergen by non-injection based means. In some embodiments, the administration includes the swallowing of the allergen, such as by oral immunotherapy (OIT) or sublingual swallow immunotherapy (SLIT-swallow) as non-limiting examples. Sublingual immunotherapy (SLIT) and sublingual spit immunotherapy (SLIT-spit) may also be used.

In one aspect, the allergen is administered as part of a pre-treatment or conditioning of a subject. In some embodiments, the administration is prior exposure of the subject to the allergen in the environment. Non-limiting examples include pre-seasonal treatment in the case of seasonal allergies, such as those resulting from plant pollens. In other embodiments, the administration is to a subject that is previously untreated by immunotherapy, such as individuals not previously treated with clinical or other intentional administration of allergen. Non-limiting examples include unsensitized individuals, such as those not previously exposed to the allergen in their environment.

In some embodiments, the allergen is formulated to be suitable for OIT such that the allergen is delivered to the small intestine. Non-limiting examples include the use of coatings that reduce or prevent degradation of the allergen in low pH environments, such as in the mammalian stomach and part of the small intestines. The allergen thus avoids exposure to acid and proteolytic digestion, thus preserving antigenic structure of the allergen and its ability to immunize in a form more similar to that of the allergen in the environment. While stable at low pH, the coating may release the allergen in higher pH environments, such as the duodenum. In other non-limiting examples, the allergen is microencapsulated by the coating, optionally where the allergen is part of, or itself coated onto, a core.

In additional embodiments, the enteric coating is applied as a water emulsion of ethylacrylate methylacrylic acid copolymer, or hydroxypropyl methyl cellulose acetate succinate (HPMAS). Other coatings may be used, including those described in JP 93059098 B; JP 83055125 B; JP 96013748 B2; and U.S. Pat. No. 4,377,568.

In an additional aspect, the invention provides for reducing immune responses to an exogenous allergen by administration of daily doses of the allergen from about 1 to about 30 times the maintenance dose used in SCIT. This is in contrast to previous uses of oral doses from about 50 to about 200 that of SCIT. Non-limiting examples of the invention include use of doses that are increased during the pre-treatment or conditioning period.

### DEFINITIONS

The term “allergen” refers to any proteinaceous molecule or complex, including a peptide or polypeptide and complexes thereof, which is capable of inducing immune responses in a subject, such as a human being.

### BRIEF DESCRIPTION OF THE DRAWINGS

FIG. 1 shows the percentage reduction in total symptom score (TSS) over placebo (during an average of 4 peak weeks in the peak ragweed pollen season) with pre-seasonal treatment. The results indicate that pre-seasonal treatment is essential for efficacy. The indicated P-values are from 2-way Anova testing of daily dosing with versus placebo.

### DETAILED DESCRIPTION OF MODES OF PRACTICING THE INVENTION

This invention provides methods of conditioning or pre-treating a subject to reduce the immune response of the subject to an allergen. The invention may be advantageously used in a variety of contexts, including seasonal allergies wherein pre-treatment or conditioning is performed before the onset of the seasonal allergy.

In some embodiments, the invention provides a method of conditioning, or pre-treating, a subject to have a reduced immune response to an allergen, such as an environmental allergen. The immune response may be symptoms of allergic rhinitis and/or allergic conjunctivitis. The method comprises administering a composition comprising an allergen to a subject for a period of more than eight weeks prior to exposure of said subject to said allergen in the subject's environment. Non-limiting examples include those where the “period of more than eight weeks” is the period immediately prior to exposure. Thus the method may be practiced during a calendar year during which seasons of one or more seasonal allergies occur such that the composition is administered for a period at least eight weeks immediately prior to the start of the season.

The invention also provides a method of reducing a subject's immune response to an allergen, such as an environmental allergen. The immune response may be symptoms of allergic rhinitis and/or allergic conjunctivitis. The method may comprise administering a first composition comprising one or more allergens to the subject for a period of more than eight weeks prior to exposure of said subject to said allergen in the subject's environment. This is during the pre-treatment or “pre-seasonal” treatment phase. The administering may be followed by maintaining administration of said one or more allergens by a second composition comprising said one or more allergens to said subject during said exposure of the subject to said allergen in the subject's environment. This is during the maintenance, or “in season”, phase. The administration of the first and second compositions may be by any non-injection based means to reduce said subject's immune response, such as symptoms of allergic rhinitis and/or allergic conjunctivitis, to the allergen. The invention of course also encompasses the actual first and second allergen containing compositions as well as their preparation and use.

Other immune responses that may be reduced by the practice of the invention include, but are not limited to, nasal stuffiness/congestion, nasal discharge/postnasal drip, nasal itching, sneezing, itchy/burning eyes, tearing/watering eyes, redness of eyes, and itchy throat and/or ears.

Again, the “period of more than eight weeks” may be the period immediately prior to exposure. Thus in cases of allergens that cause seasonal allergies, the method may be practiced during a calendar year during which seasons of one or more seasonal allergies occur such that the composition is administered for a period at least eight weeks immediately prior to the start of the season(s).

In cases of pollen related allergies, the season may be optionally defined based upon pollen levels in the air. Using ragweed pollen as a non-limiting example, pollen counts above about 20 grains/m³ is used to define the start of ragweed season. Thus in some embodiments, start of the season may be defined as two consecutive days of pollen counts above about 20 grains/m³.

In some embodiments of the invention relating to seasonal allergies, the start of the season may be determined based upon comparison to past season(s) and/or by assessment of allergen levels in the environment, such as the air or atmosphere. The allergen in the subject's environment includes, but is not limited to, airborne or atmospheric allergens such as pollens. Embodiments of the invention include those wherein the allergen is selected from ragweed pollen, grass pollen, tree pollen, birch pollen, Japanese cedar pollen, cat hair or a dust mite allergen. Additional non-limiting examples include pollen from Ulmaceae, such as American elm (Ulmus americana); Cupressaceae, such as Mountain cedar (Juniperus ashei); Betulaceae, such as Paper birch (Betula papyrifera) and Red alder (Alnus rubra); Fagaceae, such as White oak (Quercus alba) and Red oak (Quercus rubra); Aceraceae, such as Box elder (Acer negundo); Oleaceae, White ash (Fraxinus americana) and Olive (Olea europaea); Salicaciae, such as Cottonwood, East. (Populus deltoides); Moraceae, such as Mulberry (Morus rubra); Juglandaceae, such as Pecan (Carya illinoensis) and Black walnut (Juglans nigra); Platanaceae, Sycamore (Plantanus occidentalis); Johnson grass (Holcus halepensis); Bahia grass (Paspalum notatum); Bermuda grass (Cynodon dactylon); Orchard grass (Dactylis glomerata); Kentucky blue grass (Poa pratensis); Timothy grass (Phleum pratense); Rye grass (Lolium perenne); Meadow fescue grass (Festuca elatior); Red top grass (Agrostis alba); Sweet vernal grass (Anthoxanthum odoratum); Mugwort weed (Artemisia vulgaris); Short ragweed (Ambrosia artemisiifolia); Chenopod such as Russian thistle (Salsola kali), Burning bush or summer cypress (Kochia scoparia) and Lamb's quarter (Chenopodium album); Amaranth, such as Red root pigweed (Amaranthus retroflexus), Dock-knotweed family (Buckwheat family), or Red sorrel (Rumex acetosella); and Plantaginaceae, Narrow-leafed plantain (Plantago lanceolata) as well as allergens of Deuteromycetes (molds), such as those of Alternaria alternate, Cladosporium herbarum, Cladosporium cladosporioides, Penicillium chrysogenum, Aspergillus fumigatus, Epicoccum nigrum, Helminthosporium solani; cat (Felis domesticus) epithelium or dander; dog (Canis familiaris) epithelium or dander; German cockroach (Blattella germanica); American cockroach (Periplaneta americana); House dust mite (Dermatophagoides farinae and Dermatophagoides pteronyssinus). Compositions comprising one or more of these allergens, in any combination, may also be used. Where an allergen may be considered an indoor (such as aeroallergen) or otherwise non-seasonal, their inclusion in embodiments of the invention are based upon the allergen causing one or more symptoms of seasonal allergies, possibly due to combination with a seasonal allergen. A seasonal allergy (or seasonal allergic rhinitis) is one in which symptoms like inflammation and others only occur during specific times of the year.

Thus in some embodiments, the invention may be advantageously used with subject having a history of seasonal allergic rhinitis, such as to ragweed, grass, or tree pollen. The invention provides the benefit of reduces the seasonal increase of IgE in such subjects during the respective pollen season.

Administration may be by any non-injection based means, including, but not limited to, OIT, SLIT, SLIT-spit and SLIT-swallow. The administration is by use of doses and dosing regimens that result in the reduction of immune responses to the allergen in the subject.

In some embodiments, the administration of allergen is daily and begins from about 8 or more weeks before exposure of said subject to said allergen in the subject's environment. In other embodiments, the administration is about 9, about 10, about 11, or about 12 weeks prior to exposure. The administration may at other frequencies, such as once a week or more frequently.

The invention also provides particular allergen doses for use in the disclosed methods. The daily doses may be from about 1 to about 30 times the maintenance injection dose used in SCIT expressed in microgram major allergen units as defined in reference 41 herein, which is readily determined by routine methods known in the field. The dose may be from about 2 to about 25 times, from about 3 to about 10 times, from about 4 to about 15, or from about 5 to about 20 times the SCIT maintenance dose. Using ragweed pollen extract as a non-limiting example, about 40 microgram Amb a 1 major allergen or more may be used in the practice of the invention. Doses of about 10, about 15, about 20, about 25, about 30, about 35, and about 40 microgram Amb a 1 major allergen may also be used. The doses may be increased during the conditioning or pre-seasonal treatment phases of the methods described herein.

Alternatively, and with respect to ragweed, doses of about 6, about 8, about 10, about 12, about 14, about 16, about 18, about 20, about 22, to about 24 microgram Amb a 1 (a major allergenic protein of ragweed) allergen may be used. Of course use of ragweed pollen extract that is equivalent to these Amb a 1 doses may also be used.

The administering of the invention may be by use of a microencapsulated allergen. In some embodiments, the encapsulating is by use of aqueous conditions without employing non-aqueous solvents. In other embodiments, non-aqueous solvents may be used. The encapsulating may be in the form of an enteric coating that covers the allergen or composition thereof. Non-limiting embodiments include the use of an enteric coating that is stable under low pH conditions but allows release of said allergen in the duodenum. Non-limiting examples include an ethylacrylate methacrylic acid copolymer sold under the trademark Eudragit L 30D manufactured by Rohm Pharma. This has a molecular weight of about 250,000 and may be applied as a 30% aqueous solution. Alternate coatings include hydroxypropylmethyl cellulose acetate succinate and Eudragit F30D as non-limiting examples.

In some embodiments, the coating material is used in combination with a plasticizer to improve the continuity of the coating. Non-limiting examples include triethylcitrate (TEC) sold by Morfley Inc. While plasticizers can be liquid, they are not considered to be solvents because they remain within the coating material to alter its physical characteristics. Plasticizers do not act to dissolve the allergen. Of course plasticizers which dissolve or denature the allergen would not be used in the invention unless use of such modified allergens were desired.

In some embodiments, the allergen(s) are dispersed in an aqueous solution. The solution is then sprayed onto a core particle, such as a nonpareil composed of sugar and/or starch. This results in the formation of a microsphere, which may then be coated with a polymer in solution which solidifies to become acid resistant coating. A non-limiting example of the solution is a water based emulsion of the polymer. The coating should protect the allergen as it passes through the stomach and should release the allergen into the small intestines where it can act upon the lymphoid tissue. The allergen may be in the form of pollen or pollen extract, optionally in lyophilized form.

Nonpareils are small, round particles of pharmaceutically inert materials. Commercially available nonpareils include Nupareils which is sold by Ingredient Technology Corporation. In some embodiments, the nonpareils are coated with an amount of the allergen containing solution to provide a coating of 1-10% allergen by weight on a solids basis. Coating conditions and times may vary based on the apparatus and coating viscosity. In many embodiments, the coating steps are conducted at less than 50° C., such as less than 35° C.

The allergen may be used or formulated in combination with a stabilizing agent, such as one which provides physical protection for the allergen. Non-limiting examples of such agents include therapeutically inactive water soluble sugars such as lactose, mannitol and trehalose. These agents may also protect the therapeutic antigen during the coating process. In some embodiments of the invention, about 1 to about 10% polyvinylpyrrolidone is used to aid the binding of allergen to a nonpareil.

Allergen coated microspheres that have been coated may be processed by any standard methods known in the field.

Talc (up to about 3.0% of coating composition) may also be added to prevent sticking between the microsphere particles as needed. Similarly, an antifoaming agent (such as about 0.0025% of coating composition) like sorbitan sesquioleate (Nikko Chemicals Company Limited) or silicone can be included as needed.

In some embodiments of the invention, a suitable adjuvant may be used with administration of the allergen or allergen containing composition to a subject. Suitable adjuvants are known to the skilled person and may be selected as desired. The adjuvant may be formulated to be part of an allergen containing composition of the invention.

In some embodiments, the enteric coated microspheres are placed in gel capsules for oral administration to humans as a composition of the invention. Alternative formulations of the microspheres may also be used in the practice of the invention.

The invention further provides for a plurality of doses, of allergen(s) or a composition thereof, for oral administration in methods as described herein. The plurality of doses may comprise individual compositions of the same, or increasing, amounts of an allergen, such as an environmental allergen. In some embodiments, the increasing amounts of an allergen may range from about 5 to about 40 units of one or more ragweed antigen. The composition of allergen may optionally comprise ragweed pollen extract.

Having now generally described the invention, the same will be more readily understood through reference to the following examples which are provided by way of illustration, and are not intended to be limiting of the present invention, unless specified.

### EXAMPLES

### Example 1

### Overall Design of Study

Either Microencapsulated Ragweed Pollen Extract (MRPE) or placebo was administered to human subjects. Doses of MRPE were 40 Units/day or 40 Units/week. 1 A unit is about 1 microgram Amb a 1 major allergen.

Subjects in the study were 18-65 in age, with at least a 2 year history of seasonal allergic rhinitis to ragweed. They also had positive prick test (wheal diameter at least 5 mm, erythema diameter at least 15 mm); and IgE to ragweed by CAP assay of at least 0.7 kU/L. They also had no history of allergic reactions requiring hospitalization or of systemic reaction to previous immunotherapy. Subjects further had no immunotherapy for at least one year and only moderate to severe asthma.

Dosing was started 8-12 weeks prior to the ragweed pollen season and dosing ended when ragweed pollen season was over as defined below. Overall, subjects participated for up to 24 weeks plus 4 weeks of follow-up, during which, they were scored for symptoms during the 4 peak pollen weeks of the ragweed pollen season and during the entire ragweed pollen season. Components of the symptom scoring included nasal stuffiness/congestion, nasal discharge/postnasal drip, nasal itching, sneezing, itchy/burning eyes, tearing/watering eyes, redness of eyes, and itchy throat and/or ears. A “total symptom score” (TSS) was used to evaluate the treatments.

Dosing was performed according to the schedule below. Subjects received Study Drug until the ragweed pollen season was over at their location. The end of ragweed pollen season was based on the local ragweed pollen counts. Ragweed pollen season began when ragweed pollen counts were above 20 grains/m³ for 2 consecutive days and ended when ragweed pollen counts were no longer above 20 grains/m³ for two consecutive days. Following the end of ragweed pollen season, subjects returned for a termination visit. Four weeks later, they returned for a follow-up visit.

| | Group 1 | Group 2 | Group 3 |
|---|---|---|---|
| Escalation Phase | | | |
| Week 1 (Daily Dosing) | 5 Units | 5 Units | Placebo |
| Week 2 (Daily Dosing) | 10 Units | 10 Units | Placebo |
| Week 3 (Daily Dosing) | 20 Units | 20 Units | Placebo |
| Week 4 (Daily Dosing) | 40 Units | 40 Units | Placebo |
| Maintenance Phase (Part I) | | | |
| Weeks 5-8 (Daily Dosing) | 40 Units | 40 Units | Placebo |
| Maintenance Phase (Part II) | | | |
| Weeks 9-24, Day 1 | 40 Units | 40 Units | Placebo |
| Weeks 9-24, Days 2-7 | 40 Units | Placebo | Placebo |
| Week 25, Day 1 | End of | End of | End of |
| | dosing | dosing | dosing |

### Example 2

### Results

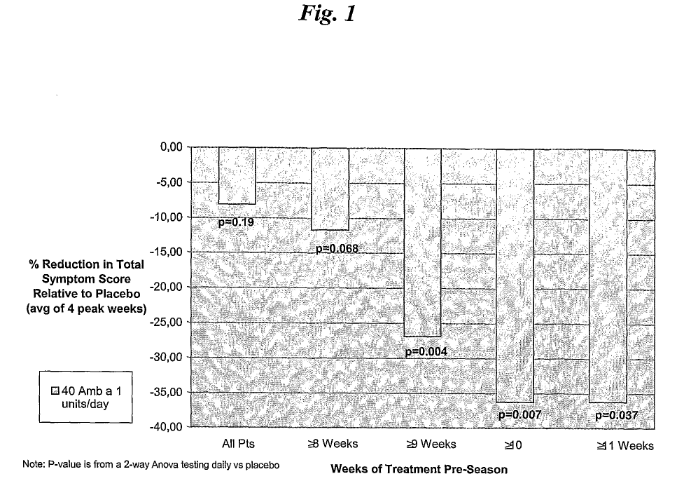

Symptom scores were analyzed for patients who had at least 8, 9, 10, 11 and 12 weeks of drug dosing prior to the ragweed season. The results are shown in FIG. 1.

Observations from the study include that the 40 Units/week dose of MRPE did not appear to affect TSS during the peak ragweed pollen season. Patients who were in the 40 Units/day and 40 Units/week groups received the same dosing regimen of MRPE for the first 8 weeks of the study. The dosing regimen consisted of a dose escalation, with daily dosing, for the first 4 weeks followed by a plateau at 40 Units/day for the subsequent 4 weeks, as shown by the table above. Beginning at Week 9, patients in the two groups received either 40 Units/day or 40 Units/week of Amb a 1. From about Week 9 on the 40 Units/week group and the placebo group are similar while the TSS in the 40 Units/day group are lower.

Patients who received less than 8 weeks of daily dosing with MRPE prior to the ragweed season showed no decrease in TSS during the 4 peak weeks of the ragweed pollen season compared to their TSS at the time of enrollment. In patients who received Study Drug for at least 9 weeks prior to the ragweed season, the difference between the 40 Units/day group and placebo was statistically significant.

The average improvement in TSS over placebo for the four peak pollen weeks are shown in FIG. 1. Initiating treatment with MRPE at least 9 weeks of prior to the ragweed season gives a 30-40% reduction in total symptom score relative to placebo.

The immunology data from these studies show consistently that ragweed specific IgG was increased whereas the seasonal related increase in IgE is blunted on exposure to MRPE. The analysis clearly demonstrates the importance of initiating treatment well in advance of the ragweed season. A dose of 40 Amb a 1 Units/day has been shown statistically to significantly reduce the rhinoconjunctivitis symptoms with about 39% over placebo when patients are pretreated with MPRE for at least 10 weeks prior to the ragweed season.

### REFERENCES

1 Litwin et al. “Oral immunotherapy with short ragweed extract in a novel encapsulated preparation: a double-blind study” J Allergy Clin Immunol. 1997 July; 100(1):30-8.
2 Van Deusen et al. “Efficacy and safety of oral immunotherapy with short ragweed extract” Ann Allergy Asthma Immunol. 1997 June; 78(6):573-80.
3 Ledford, D K “Is oral immunotherapy too good to be true? Ann Allergy Asthma Immunol. 2004 January; 92(1):1-2.
4 TePas et al. “Clinical efficacy of microencapsulated timothy grass pollen extract in grass-allergic individuals.” Ann Allergy Asthma Immunol. 2004 January; 92(1):25-31.
5 Litwin et al. “Immunologic effects of encapsulated short ragweed extract: a potent new agent for oral immunotherapy” Ann Allergy Asthma Immunol. 1996 August; 77(2): 132-8.
6 Bagnasco et al. “Absorption and distribution kinetics of the major Parietaria judaica allergen (Par j 1) administered by noninjectable routes in healthy human beings” J Allergy Clin Immunol. 1997 July; 100(1):122-9.
7 Walter et al. “Functionalisation of allergen-loaded microspheres with wheat germ agglutinin for targeting enterocytes.” Biochem Biophys Res Commun. 2004 Mar. 5; 315(2):281-7.
8 Roth-Walter et al. “M cell targeting with Aleuria aurantia lectin as a novel approach for oral allergen immunotherapy.” J Allergy Clin Immunol. 2004 December; 114(6):1362-8.
9 Wang et al. “Management of allergic rhinitis: a common part of practice in primary care clinics.” Allergy. 2004 March; 59(3):315-9.
10 Canonica et al. “Noninjection routes for immunotherapy.” J Allergy Clin Immunol. 2003 March; 111(3):437-48.
11 Patriarca et al. “Oral desensitizing treatment in food allergy: clinical and immunological results.” Aliment Pharmacol Ther. 2003 February; 17(3):459-65.
12 Oppenheimer et al. “Safety and efficacy of oral immunotherapy with standardized cat extract” J Allergy Clin Immunol. 1994 January; 93(1 Pt 1):61-7.
13 Swarbrick et al. “Absorption of antigens after oral immunisation and the simultaneous induction of specific systemic tolerance.” Gut. 1979 February; 20(2):121-5.
14 Cooper et al. “A controlled trial of oral hyposensitization in pollen asthma and rhinitis in children.” Clin Allergy. 1984 November; 14(6):541-50.
15 Giovane et al. “A three-year double-blind placebo-controlled study with specific oral immunotherapy to Dermatophagoides: evidence of safety and efficacy in paediatric patients.” Clin Exp Allergy. 1994 January; 24(1):53-9.
16 Mosbech et al. “High dose grass pollen tablets used for hyposensitization in hay fever patients. A one-year double blind placebo-controlled study.” Allergy. 1987 August; 42(6):451-5.
17 Moller et al. “Oral immunotherapy of children with rhinoconjunctivitis due to birch pollen allergy. A double blind study.” Allergy. 1986 May; 41(4):271-9.
18 Moller et al. “Oral immunotherapy of children with rhinoconjunctivitis due to birch pollen allergy. A double blind study.” Allergy. 1986 May; 41(4):271-9.
19 Taudorf et al. “Oral administration of grass pollen to hay fever patients. An efficacy study in oral hyposensitization.” Allergy. 1985 July; 40(5):321-35.
20 Litwin et al. “Microencapsulation: The future of oral immunotheraphy?” BioDrugs 1998 Apr. 9 261-270.
21 Barone et al. “Abrogation of oral tolerance by feeding encapsulated antigen.” Cell Immunol. 2000 Feb. 1; 199(2):65-72.
22 Mayer et al. “Therapeutic potential of oral tolerance.” Nat Rev Immunol. 2004 June; 4(6):407-19.
23 “Induction of oral tolerance in TGF-beta 1 null mice.” J Immunol. 1998 Jul. 1; 161(1):154-60.
24 Jain et al. “Activation patterns of murine B cells after oral administration of an encapsulated soluble antigen.” Vaccine. 1996 September; 14(13):1291-7.
25 Hong et al. “Pepsin-digested peanut contains T-cell epitopes but no IgE epitopes.” J Allergy Clin Immunol. 1999 August; 104(2 Pt 1):473-8.
26 Taudorf et al. “Oral immunotherapy in birch pollen hay fever.” J Allergy Clin Immunol. 1987 August; 80(2):153-61.
27 Bagnasco et al. “Pharmacokinetics of an allergen and a monomeric allergoid for oromucosal immunotherapy in allergic volunteers.” Clin Exp Allergy. 2001 January; 31(1):54-60.
28 Van Wilsem et al. “Dendritic cells of the oral mucosa and the induction of oral tolerance. A local affair.” Immunology. 1994 September; 83(1):128-32
29 Marcucci et al. “Sublingual tryptase and ECP in children treated with grass pollen sublingual immunotherapy (SLIT): safety and immunologic implications.” Allergy. 2001 November; 56(11):1091-5.
30 Bousquet et al. “Characteristics of patients with seasonal allergic rhinitis and concomitant asthma.” Allergy. 2004 June; 34(6):897-903.
31 Canonica et al. “Sublingual and oral immunotherapy.” Immunol Allergy Clin North Am. 2004 November; 24(4):685-704.
32 Canonica et al. “Sublingual and oral immunotherapy.” Immunol Allergy Clin North Am. 2004 November; 24(4):685-704, vii.
33 Passalacqua et al. “Articles 3 Oral and sublingual immunotherapy in paediatric patients.” Curr Opin Allergy Clin Immunol. 2003 April; 3(2):139-45.
34 Guerin et al. “Microsphere entrapped bee-venom phospholipase A2 retains specific IgE binding capacity: a possible use for oral specific immunotherapy.” J Microencasul. 2002 November-December; 19(6):761-5.
35 Criado Molina et al. “Immunotherapy with an oral Alternaria extract in childhood asthma. Clinical safety and efficacy and effects on in vivo and in vitro parameters” Allergol Immunopathol (Madr). 2002 November-December; 30(6):319-30.
36 Martorell Aragones “New administration routes for immunotherapy. A. Allergol Immunopathol (Madr). 2000 May-June; 28(3):93-102.
37 Creticos et al. “Responses to ragweed-pollen nasal challenge before and after immunotherapy” J. Allergy Clin Immunol. 1989 August; 84(2):197-205.
38 Creticos et al. “Efficacy safety and kinetics of oral ragweed immunotherapy in the treatment of allergic seasonal rhinitis” J. Allergy Clin Immunol. 1990; 85(1, part 2):165, abstract 85.
39 Aramaki et al. “Induction of oral tolerance after feeding of ragweed pollen extract in mice” Immunol. Lett. 1994; 40(1):21-25.
40 Li et al. “Allergen immunotherapy: a practice parameter”. Ann. Allergy Asthma Immunol. 2003; 90:1-40.
41 Nelson, “The use of standardized extracts in allergen immunotherapy”. J. Allergy Clin. Immunol. 2000; 106 (1): 41-45.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A method of conditioning a subject to have reduced symptoms of allergic rhinitis or allergic conjunctivitis to an environmental allergen, said method comprising
non-injection based administration of a composition comprising an allergen to said subject, such that said allergen is delivered to the small intestine, for a period of more than eight weeks prior to exposure of said subject to said allergen in the subject's environment,
wherein said administration of said composition conditions said subject to have reduced symptoms of allergic rhinitis or allergic conjunctivitis to said environmental allergen.

2. The method of claim 1 wherein said environmental allergen is present in ragweed pollen, is present in grass pollen, is present in tree pollen, is present in birch pollen, is present in Japanese cedar pollen, is present in cat hair, or is a dust mite allergen.

3. The method of claim 1 wherein said allergen is present in ragweed pollen, is present in grass pollen, is present in tree pollen, is present in birch pollen, or is present in Japanese cedar pollen, and said exposure is to pollen in said subject's environment.

4. The method of claim 1 wherein said administration is more than once a week, preferably daily, and begins 9 weeks or more weeks before exposure of said subject to said allergen.

5. The method of claim 2 wherein said subject has a history of seasonal allergic rhinitis or conjunctivitis.

6. The method of claim 2 wherein said administering reduces the seasonal increase of IgE in said subject during pollen season.

7. The method of claim 1 wherein said composition comprises doses of from about 1 to about 30 times the maintenance injection dose used in SCIT expressed in microgram major allergen units optionally increasing during said period.

8. The method of claim 1 wherein said composition comprising an allergen further comprises an enteric coating.

9. The method of claim 8 wherein said enteric coating is stable under low pH conditions but allows release of said allergen in the duodenum.

10. The method of claim 1 wherein said non-injection based administration is selected from oral administration and SLIT-swallow.

11. A method of reducing a subject's immune response to an allergen, said method comprising
oral based administration of a first composition comprising one or more allergens to said subject, such that said allergens are delivered to the small intestine, for a period of more than eight weeks prior to exposure of said subject to said allergen in the subject's environment, and
maintaining oral administration of said one or more allergens by a second composition comprising said one or more allergens to said subject during said exposure of the subject to said allergen in the subject's environment,
wherein said administrations of said first and second compositions reduces said subject's immune response to the allergen.

12. The method of claim 11 wherein said first composition comprises ragweed pollen allergen, grass pollen allergen, tree pollen allergen, birch pollen allergen, Japanese cedar pollen allergen, cat hair allergen or a dust mite allergen.

13. The method of claim 12 wherein said first composition comprises ragweed pollen allergen, grass pollen allergen, tree pollen allergen, birch pollen allergen, Japanese cedar pollen allergen.

14. The method of claim 11 wherein said allergen is present in ragweed pollen extract and said exposure is to ragweed pollen in said subject's environment.

15. The method of claim 11 wherein said administration is more than once a week, preferably daily, and begins 9 weeks or more weeks before exposure of said subject to said allergen.

16. The method of claim 11 wherein said subject has a history of seasonal allergic rhinitis or conjunctivitis.

17. The method of claim 13 wherein said administering reduces the seasonal increase of IgE in said subject during pollen season.

18. The method of claim 11 wherein said composition comprises doses of from about 1 to about 30 times the maintenance injection dose used in SCIT expressed in microgram major allergen units, optionally increasing during said period.

19. The method of claim 11 wherein said composition comprising one or more allergens further comprises an enteric coating.

20. The method of claim 19 wherein said enteric coating is stable under low pH conditions but allows release of said allergen in the duodenum.

21. (canceled)

22. A plurality of doses for oral administration, said plurality of doses comprising individual compositions of increasing amounts, ranging from about 5 to about 40 units of one or more ragweed antigen.

23. The plurality of doses of claim 22, wherein said compositions comprise ragweed pollen extract.

24. The method of claim 11, wherein the oral administration is for a period of about 10 weeks, about 11 weeks or about 12 weeks prior to exposure.

25. The method of claim 11, wherein the allergen in the subject's environment is an environmental allergen.

26. The method of claim 11, wherein said immune response to be reduced is symptoms of allergic rhinitis or allergic conjunctivitis.
